(19) United States
(12) Patent Application Publication
(10) Pub. No.: US 2022/0083946 A1
(43) Pub. Date: Mar. 17, 2022

(54) POST-HARVEST OPTIMIZATION

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/240,090

(22) Filed: Apr. 26, 2021

Related U.S. Application Data
(63) Continuation of application No. PCT/IB2019/058966, filed on Oct. 22, 2019.
(60) Provisional application No. 62/750,125, filed on Oct. 24, 2018.

(51) Int. Cl. G06Q 10/06; G06Q 50/02; G06F 16/2457

(52) U.S. Cl. CPC G06Q 10/06393; G06F 16/2457; G06Q 50/02; G06Q 10/0635

(57) ABSTRACT

Methods and apparatus consistent with the present disclosure may provide information to growers of Cannabis plants such that Cannabis plant biomass extractors could optimize process parameters based on growing and post processing conditions of a batch of Cannabis plants. Parameters associated with the growth and processing of Cannabis, include light, nutrition, water, humidity. Each of these parameters may affect the size, density, moisture content, cannabinoid content, or other features of a Cannabis biomass. These methods and apparatus may also help farmers protect their crop from damage by mold infestation, for example or from theft. Methods consistent with the present disclosure may analyze data from a set of sensors when Cannabis plant biomass is prepared for extraction. These sensors may collect data from the processing steps of cutting, trimming, drying, and curing when Cannabis plant matter is prepared for extraction.

### CROSS-REFERENCE TO RELATED APPLICATIONS

The present application is a continuation of International Application No. PCT/IB2019/058966 filed Oct. 22, 2019, which claims priority benefit of U.S. provisional patent application Ser. No. 62/750,125 filed on Oct. 24, 2018, the disclosures of which are incorporated, herein, by reference.

### BACKGROUND OF THE INVENTION

### 1. Field of Invention

The present disclosure is generally related to collecting and evaluating plant processing data when identifying processing risks or when identifying whether the processing of a current batch of Cannabis plant material is consistent with earlier batches of Cannabis plant material. More specifically, the present disclosure relates to the use of sensors for quality tracking during harvest and post-harvest plant processing and to identifying whether the processing of a current batch of plant matter is consistent with conditions that could help optimize process efficiencies and yields.

### 2. Description of the Related Art

The term Cannabis or “Cannabis biomass” encompasses the Cannabis sativa plant and also variants thereof, including subspecies Sativa, Indica and Ruderalis, Cannabis cultivars, and Cannabis chemovars (varieties characterised by chemical composition), which naturally contain different amounts of the individual cannabinoids, and also plants which are the result of genetic crosses. The term “Cannabis biomass” is to be interpreted accordingly as encompassing plant material derived from one or more Cannabis plants.

Cannabis biomass contains a unique class of terpeno-phenolic compounds known as cannabinoids or phytocannabinoids. Similarly, the Cannabis plant may contain a plurality of terpene, terpenoid or phenolic compounds which may impart their own therapeutic or organoleptic properties to the plant, or may act synergistically with cannabinoids and other components to provide certain effects. The proportion of cannabinoids and other compounds in a Cannabis plant may depend upon soil, climate, harvesting time and methods and post-harvesting processing handling. Post harvesting processing and handling may include for example physical separation of flowers, leaves and trim, drying, curing or decarboxylation conditions, and storage and shipping conditions.

Cannabis extracts may be obtained from Cannabis biomass by any number of methods, including but not limited to supercritical fluid extraction, solvent extraction of microwave-assisted extraction. In most cases, the yield and quality of Cannabis extract obtained will depend upon the composition of the Cannabis biomass used for the extraction, including for example the potency or concentration of cannabinoids present in the Cannabis biomass. In some cases, the yield of Cannabis extract and the quality of Cannabis extract may be different depending on the extraction conditions used to obtain the extract, for example solvent type, ratio of solvent to biomass, temperature and time of extraction, etc. The quality of the Cannabis extract may be dictated by the potency or concentration of cannabinoids in the extract, or the cannabinoid profile in the extract (i.e. the relative concentrations of various cannabinoids present), or the terpene profile in the extract (i.e. relative concentrations of various terpenes present). The quality of the Cannabis extract may also be dictated by the physical properties of the extract, including but not limited to color or viscosity.

Cannabis harvest and post-harvest handling is key aspect to obtaining high Cannabis extract yield and quality. Therefore, there is a high need for tracking the harvest and post-harvesting processing (e.g. handling, drying, storing, and transportation of biomass) so that changes in potency and quality can be detected and communicated to growers and extractors.

Growers lack extraction information when trying to correlate growth parameters with biomass extraction efficiency. In many cases a seed to sale tracking system that incorporates sensor data related to the harvest part of the supply chain process is preferred. This may include video analysis of the part of the plant harvested, in order to identify damage, theft, or other quality control issues. Moreover, information that ties specific biomass attributes with previous batches of Cannabis plant biomass may help improve methods used to grow, process, and extract elements from Cannabis plants. Therefore, there is a need for providing growers with information about how these growers can grow and process their Cannabis plants in ways that help reduce extraction costs and that optimizes the quality of concentrate produced during an extraction process. There is also a need to identify the extraction parameters that may be used to produce high quality extracts based on historical data.

### SUMMARY OF THE CLAIMED INVENTION

The presently claimed invention relates to a method, a non-transitory computer readable storage medium, or an apparatus executing functions consistent with the present disclosure. A method consistent with the present disclosure may include receiving sensor data from sensors that sense data during one or more plant matter processing steps and retrieving extraction data from a database. This method may also include performing an analysis that identifies one or more metrics, evaluates the one or more metrics, and identifies that the one or more metrics are consistent with the retrieved extraction data.

When the presently claimed invention is implemented as a non-transitory computer-readable storage medium, a processor executing instructions out of a memory may perform a method consistent with the present disclosure. Here again the method may include receiving sensor data from sensors that sense data during one or more plant matter processing steps and retrieving extraction data from a database. This method may also include performing an analysis that identifies one or more metrics, evaluates the one or more metrics, and identifies that the one or more metrics are consistent with the retrieved extraction data.

An apparatus consistent with the present disclosure may include one or more sensors, a memory, and a processor that executes instructions out of the memory. The processor when executing the instructions out of the memory may receive sensor data from the one or more sensors during the one or more plant matter biomass processing steps, retrieve extraction data from an extraction database, identify one or more metrics based on an analysis of the received sensor data received from the one or more sensors during the one or more plant mater biomass processing steps, evaluate the one or more metrics and the extraction data retrieved from the extraction database, and identify that the one or more metrics are consistent with extraction data retrieved from the extraction database.

### BRIEF DESCRIPTIONS OF THE DRAWINGS

FIG. 1 illustrates an exemplary network environment in which a system for tracking harvest and extraction of Cannabis plants may be implemented.

FIG. 2 is a flowchart illustrating an exemplary method for harvesting and preparing Cannabis plant matter for extraction.

FIG. 3 is a flowchart illustrating an exemplary method for tracking harvest data.

FIG. 4 is a flowchart illustrating an exemplary method for harvest data analytics.

FIG. 5 is a flowchart illustrating an exemplary method for identifying a quality of a set of Cannabis plant matter.

FIG. 6 is a flowchart illustrating an exemplary method for identifying a damage metric of a set of Cannabis plant matter.

FIG. 7 is a flowchart illustrating an exemplary method for identifying a theft metric of a set of Cannabis plant matter

FIG. 8 is a flowchart illustrating an exemplary method for identifying a extraction metrics of a set of Cannabis plant matter.

FIG. 9 illustrates a computing system that may be used to implement an embodiment of the present invention.

### DETAILED DESCRIPTION

The present disclosure is directed to improving the harvesting and post-harvesting operations of Cannabis plant matter. An apparatus consistent with the present disclosure may include a harvesting platform computer (e.g., server) that monitors data regarding all operations involved in harvesting and preparing Cannabis plants for delivery to extractors. Harvesting and post-harvesting may include fan leave removal, wet trimming, drying, de-stemming, curing, and shipping. Alternatively, downstream plant processing could include such fan leave removal, drying, de-stemming, dry trimming, curing, and shipping.

Note that a harvesting platform server may be coupled to sensors of a sensor platform. Such a sensor platform may include multiple and different sensors at each of a series of processing steps. Some of these sensors may be involved in all steps of the post-harvest processing (e.g. hyperspectral cameras, imaging sensors) and other sensors may only be involved in a few steps of the post-harvest processing (e.g. mold sensors).

Methods and apparatus consistent with the present disclosure may provide information to growers of Cannabis plants such that Cannabis plant biomass extractors could optimize process parameters based on growing and post processing conditions of a batch of Cannabis plants. Parameters associated with the growth and processing of Cannabis, include for example light, nutrition, water, humidity. Each of these parameters may affect the size, density, moisture content, cannabinoid content, or other features of a Cannabis biomass. Such parameters may be tracked and analyzed to identify recommendations to help farmers enhance and protect their specific Cannabis crops (e.g., from damage by mold infestation or from theft). Note that for the purposes of the present disclosure, a farmer may be any grower of Cannabis (e.g. at a farm, a greenhouse, or an indoor growing facility). Methods consistent with the present disclosure may analyze data from a set of sensors when sets (e.g., specific lots) of Cannabis plant biomass are prepared for extraction. These sensors may collect data regarding such sets of biomass at various stages, including during cutting, trimming, drying, and curing.

Additionally, such collected data may be used to generate recommendations for both growers and extractors of biomass to enhance and control quality of the biomass and end product. These methods and apparatus may also help correlate harvest conditions with extraction efficiency metrics that could potentially lead to reducing costs and that could improve supply chain efficiencies.

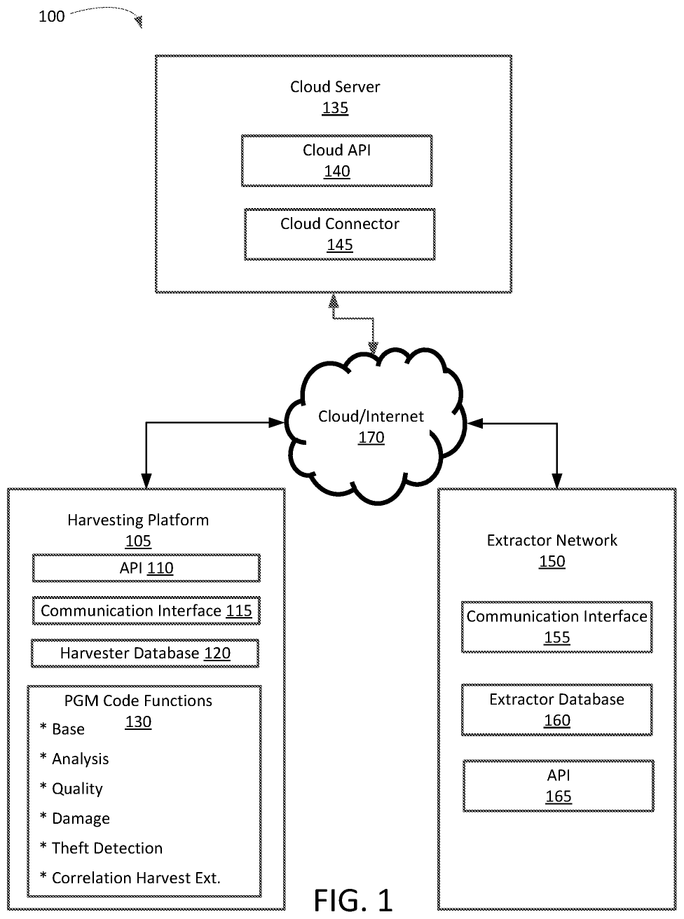

FIG. 1 illustrates an exemplary network environment in which a system for tracking harvest and extraction of Cannabis plants may be implemented. FIG. 1 includes harvesting platform server 105, cloud server 135, and extractor network 150 that may communicate with each other via the cloud or Internet 170. Harvesting platform server 105 includes application program interface (API) 110, communication interface 115, harvester database 120, and program code functions 130. Cloud server 135 may communicate with harvesting platform server 105 or extractor network 150 via cloud connector 145 to provide program code associated with cloud API 140 to harvesting platform server 105 or to extractor network 150. As such cloud server 135 may be a server that provides program applications to computing devices in a manner similar to how applications are downloaded from an online application store. Extractor network 150 includes communication interface 155, extractor database 160, and API 165.

Program code functions 130 of harvesting platform server 105 may include software functions or modules that may be referred to as a base module, an analysis module, a quality module, a damage module, a theft detection module, and a correlation harvest extraction module. Harvester database 120 may store updated sensor data continuously. This sensor data may include various metrics (e.g. color, weight, or humidity) corresponding to stages of the harvesting platform server 105 when plants are cut, dried, trimmed, or cured. Harvester database 120 may also store updates to the different metrics from the different software modules and may store quality metrics (indicating a high, a medium, or low plant matter quality), damage metrics (indicating a high, a medium, or a low amount of plant matter damage), and a theft metric (indicating a high, medium, low amount of theft). The quality metrics, damage metrics, and the theft metrics stored in the harvester database 120 may have been identified by a processor executing instructions of the quality software module, the damage software module, and the theft detection module. After the quality, damage, and theft metrics have been collected and stored, they may be evaluated by a processor executing instructions consistent with the correlation harvest extraction software module to correlate harvest data to extraction metrics or extraction predictions. These steps that may be used to assign a high, a medium, or a low extraction efficiency to a batch of Cannabis plant matter. The correlation harvest to extraction module may also initiate an alert or warning message, when appropriate. For easiness of representation the harvesting database may be divided into two databases—one related to the cutting to drying steps of post-harvesting handling and one related to de-stemming and shipping steps of a process that prepares the plant matter for chemical extraction. The harvesting database 120 may also store data related to the cutting to drying steps of Cannabis plant processing after the plants have been harvested. The harvesting database 120 may store data related to a series of process steps that may begin with a de-stemming step and that complete with a shipping step. As such, software of the harvesting platform server 105 may pull or receive data from sensors communicatively coupled to the harvesting platform server 105. Data from each step post-harvest may be stored at harvesting database 120 continuously as sensor data is received. This how data stored at the harvesting database 120 may be updated with new sensor data over time. The processor at harvesting platform server 105 may allow extractor network computer 150 to access data stored at the harvesting database 120 via communication interface 115 and API 110. As such, extractor network computer 150 may pull data out of the harvesting database 120. In certain instances, data stored at extractor database 160 may be accessed by harvesting platform server 105. Communications between harvesting platform server 105 and extractor network 150 may be performed using communication interface 155 and API 165 at extractor network 150 and may be performed using communication interface 115 and API 110 at harvesting platform server 105. Communications between harvesting platform server 105 and extractor network 150 may allow sensor data and extractor data to be shared such that sets of program code at harvesting platform server 105 or extractor network 150 may analyze harvesting data and extraction data when generating reports or when generating warnings/alerts based on results from an analysis. In certain instances, the analysis software module at harvesting platform server 105 may perform this analysis. In operation this analysis software may receive sensor data and extractor data from the harvesting base module and provide that data for use by the quality module, the damage module, the theft module, and the correlation harvest to extractor module. The operation of these different software functions or modules may collect results that may include quality metrics, damage metrics, and theft metrics and generate warnings based on these results. In certain instances, these results may be sent to the harvesting base module for storage at the harvesting database 105. The quality module may receive sensor data and extractor data from the analysis module continuously such that the analysis module may estimate the color and density of plant material as it is processed. These results, colors, and densities may be compared to historical values of successful runs by an extractor who controls extractor database 160. Based on the value of that comparison, a quality metric may be estimated (e.g. high, medium, low). If any of the sensor values is below a given value, a “Yes” status or value is given for the warning parameter and a warning may be issued. The metric and warning value may then be sent back to the analysis module.

For example, the temperature and humidity of each stage of plant processing may be tracked by the harvesting platform server 105 factors that may affect the quality of the Cannabis biomass are monitored. Here the temperature and humidity data collected during different steps of a harvesting/post-harvesting process may be monitored and evaluated. The harvesting platform server 105 may identify that the temperature always stays around 68 F (20 C) and identify that the humidity is maintained around 45%. The extractor historical database 160 may store data that indicates that when temperatures stay around 68 F and humidity stays around 45% across each of the post-harvesting steps, the efficiency of the extraction should be high and this may result in the quality metric also being high. In such an instance, no warning may need to be generated. In instances when the extractor historical database 160 stores data that indicates when the temperature is higher than 80 F or lower than 40 F or the humidity is above 70% or lower than 20%, a warning message should be generated. This warning message may inform a harvester that a batch of Cannabis plant material may be assigned a low quality metric if these conditions persist longer than a threshold amount of time.

The damage module may also receive sensor data and extractor data from the analysis module continuously when estimates regarding the color and density of plant matter are collected and compared to historical data of successful extraction runs stored in the extractor database 160. Based on a value of that comparison, a damage metric may be estimated (e.g. high, medium, low). If either of the sensor values is below a given threshold value, a “Yes” value is given for the warning parameter. The metric and warning values may then be sent back to the analysis module and a warning message may be generated.

In another example, mold is one parameter that could damage the flowers of Cannabis plant material. Since mold may grow during a wet trimming process, the damage module may receive mold sensor data from the wet trimming phase. A processor executing instructions of the damage module when an average mold grow rate is calculated. In such an instance, the mold sensor data may identify that mold should grow by a value of 3 mm/day. Data stored in the extractor historical database 160 may indicate that when the mold grow rate in the wet trimming phase is higher than 5 mm/day, the efficiency of the extraction reduces. This data may also identify that when the mold grow rate is between 2 mm/day and 5 mm/day, extraction efficiency is average. The data stored at the extractor data base 160 may also indicate that when the grow rate is below 2 mm/day, the extraction efficiency is higher. That means that the damage metric for this example may be medium and so the wet trimming can proceed with no warning messages being sent or alarms being triggered.

The theft module may also receive sensor data and extractor data from the analysis module continuously when the theft module estimates the weight of plant matter and compares those weight values to historical values of successful processing runs stored in the extractor database 160. Based on the value of that comparison, a theft metric may be estimated as high, medium, or low. Theft may be detected when a weight of Cannabis plant matter changes more than a threshold amount beyond a value of an expected weight change. For example, historical data may identify that Cannabis plants of a certain type reduce in weight over time based on drying or curing conditions or weights measured when dried and cured plant matter are shipped. If a difference in weight is identified as being higher than a given threshold value, a “Yes” value may be assigned to a warning parameter. Such warning parameters and related metrics may then be sent back to the analysis module.

As an example, theft may happen between the curing and the shipping steps. The theft module receives weight sensor data at the end of the curing process and at the beginning of the shipment. The theft module calculates the average % of weight lost in the transfer—0.3% in this example. The extractor historical database 160 may store data that indicates that 0.5% of weight loss may be expected during transfer from curing to shipping and that that loss would not affect the efficiency of the extraction. Therefore, the theft metric should be high, and no warning should be generated. The correlation harvest to extraction module may also receive sensor data and extractor data from the analysis module continuously. Here again estimates associated with the color and density of plant matter may be compared historical values of successful runs stored in the extractor database 160. Based on the value of that comparison, a correlation harvest to extraction metric may be estimated as high, medium, or low. If either of the sensor values is below a given value, a ““Yes”” value is given for the theft warning parameter and the metric and warning values may then be sent back to the analysis module.

As an example, changes in color during curing may indicate over curing. The correlation harvest to extraction module may receive color data from the hyperspectral camera placed at the curing step. This correlation and extraction module may identify whether similar data found for the curing process is stored at the extractor historical database 160. The data stored at the historical database 160 may indicate that for similar biomass, colors in the green spectrum during curing show a decrease in extraction efficiency and therefore, if the data from the hyperspectral camera shows the color of the Cannabis biomass falling into the green range of wavelengths, a “Yes” value may be assigned to the warning parameter. The harvesting base module would generate a warning for the harvester to stop the curing process based on this warning. In certain instances, the warning may be sent via a piece of hardware (such as communication interface 115) that may be capable of transmitting an analog or digital signal over the telephone, other communication wire, or wirelessly within the harvesting platform. This warning and related data may also be sent to the extractor network 150.

Extractor network 150 may collect, track, and store extraction data in the extractor database 160. This extractor data may be sent to the harvesting platform server 105 by via API and 110 and communications interface 115. Historical database 160 may store data that keeps track of the incoming material (such as a Biomass Number), extraction parameters (such as solvent type, microwave energy level, extraction temperatures, a ratio of solvent volume to plant mass, or extraction time, e.g. length of time that Cannabis biomass resides (residence time) in a continuous flow extraction chamber)), and extraction results (such as an efficiency rate, weight percentage content of tetrahydrocannabinol (THC) or cannabidiol (CBD)). Communication interface 155 at extractor network 150 may transmit an analog or digital signal over the telephone or other communication wire. Alternatively, communication interface 155 may be a wireless interface that sends data wirelessly to the harvesting platform server 105.

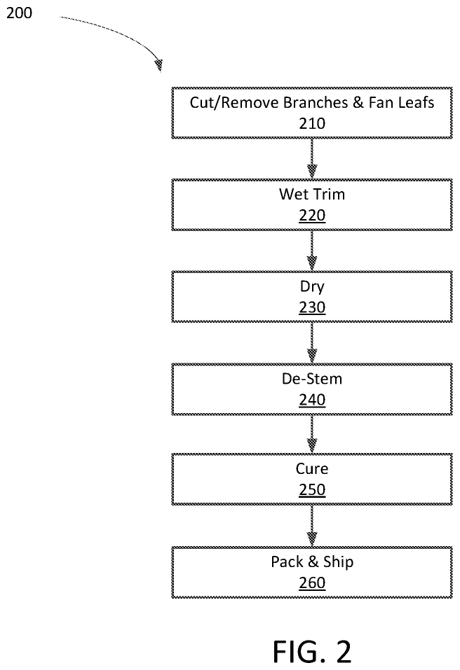

FIG. 2 is a flowchart illustrating an exemplary method 200 for harvesting and preparing Cannabis plant matter for extraction. Sensors associated with a harvesting platform like the harvesting platform server 105 of FIG. 1 allows for data to be collected relating to the processing of Cannabis plant matter after the plant matter has been harvested. The processing of the Cannabis plant matter begins with step 210 of FIG. 2 where the branches are cut off the main stems of the Cannabis plants. Step 210 may also include cutting fan leafs off stems of the Cannabis plants. Next, is optional step 220 of FIG. 2 where the Cannabis plants may be trimmed before the plants are dried. Wet trimming may include trimming of trimming the leaves off of the Cannabis flowers immediately after the branches are cut from the main plant stems. Since most mold can occur during this wet trimming step 220, mold sensors may be used during this step to track the presence and quantity of mold. In other instances, dry trimming may be performed after the branches are dried in step 230 of FIG. 2.

Whether or not wet trimming is performed, larger fan leafs may be cut off the Cannabis plant stems immediately before drying step 230. In this process, growers may cut off and dry “sugary” fan leafs. These “sugary” fan leafs may be dried in the same environment as Cannabis flowers. The term “sugary” refers to leafs that have trichomes that causes these leafs to be sticky to the touch. In instances when leafs in and around Cannabis flowers are not trimmed off in wet trimming step 220, the leafs attached to the Cannabis flowers may curl up closer the Cannabis flowers. Drying the Cannabis plants may be performed either after wet trimming step or right after the large fan leaves have been removed (if the grower is opting for a dry trim method).

In certain instances whole Cannabis plants after being cut down may be hung upside down to dry, this process may include cutting branches into smaller, more manageable sections (branches) or left as an entire plant during drying step 230. After the Cannabis plant matter has been dried, de-stemming may be performed in step 240 of FIG. 2. De-stemming step 240 may include removing stems from the Cannabis flowers. The process of de-stemming may produce a plurality of flowers that do not contain bare stems. Next, the Cannabis flower may be cured, this curing process involves drying the Cannabis flowers very slowly to enrich the flowers' flavor. Temperature, moisture, UV light and oxygen levels are important parameters in this process. It is better when containers used for the curing process are stored in a cool, dark place where they can be examined daily. For the first week or two, the containers should be opened (“burped”) once or twice a day. This lets out some of the built up humidity and allows some fresh air in, at curing step 250. Alternatively, dry air may be gently circulated through the plant matter during curing step 250. Next, in step 260, the Cannabis cured Cannabis plant matter may be packaged and shipped from a plant processing center to an extraction facility.

Table 1 identifies sensors from which data may be collected during the different processing steps of FIG. 2. The X marks in table 1 identifies sensors that may be used to monitor plant processing at each step of FIG. 2. Note that some of the sensors in FIG. 2 are involved in all steps of the harvesting platform (e.g. hyperspectral cameras, imaging sensors, & weight/density sensors), where other sensors are only involved in a few steps of the harvesting platform (e.g. mold sensors, heat and temperature sensors for curing and drying steps). Note that most of the sensors identified in table 2 are not used in the many of the processing steps of FIG. 2. The curing step, however, uses all of the sensors identified in table 2 except for mold sensors. This is because the environmental sensors used during the curing step may help prevent mold growth by triggering control of humidity and temperature.

TABLE 1
Sensor Platform Data Collection

| | Trim Fan | Wet | | De- | | |
|---|---|---|---|---|---|---|
| Sensor | Leafs | Trim | Dry | Stem | Cure | Shipping |
| Color | X | X | X | X | X | X |
| (hyperspectral | | | | | | |
| camera) & | | | | | | |
| Imaging | | | | | | |
| Weight/Density | X | X | X | X | X | X |
| Mold | | X | | X | | X |
| Temperature | | | X | | X | |
| Humidity | | | X | | X | |
| Time | | | | | X | |
| Heat | | | | | X | |
| Oxygen | | | | | X | |

Table 2 includes data that may have been collected or generated in each of steps 210, 220, and 230 of FIG. 2 when lots of Cannabis plants 55 and 56 were processed. Table 2 includes a first set of data collected or generated during the cutting step 210, a second set of data collected or generated during the wet trimming step 220, and a third set of data collected during the drying step 230 of FIG. 2. The data included in table 2 may be stored in harvesting database 120 of FIG. 1. Sensor data collected during each of these steps may have been collected and evaluated continuously when the content of table 2 was added to table 2. Table 2 includes average colors, plant densities, metrics (e.g. quality, damage, theft, & correlation/extraction metrics), alert levels (e.g. quality, damage, theft, correction/extract alert levels), and alert status. Note that levels assigned to the quality metric, the damage metric, the theft metric, and the correlation/extraction (Corr./EXT) metric may have a value of high, medium, or low, for example. A high metric may identify that a plant quality, an amount of damage, an amount of theft, or and expected extraction efficiencies for a given lot of Cannabis plant material are each at a preferred level. As such, high metrics may identify that the Cannabis plant material is at a high quality level, an amount of damage is little to none, an amount of theft is little to none, and that the Cannabis plant material has been identified to have factors that are consistent with Cannabis plant matter that was previously extracted. Medium metrics may identify that the Cannabis plant material quality is at a medium level, an amount of damage is modest, an amount of theft is modest, and that the Cannabis plant material has been identified to have factors that are similar to, yet not judged to be completely consistent with Cannabis plant matter that was previously extracted. Low metrics may identify that the Cannabis plant material quality is at a low level, an amount of damage is above a threshold level, an amount of theft is above a threshold level, and that the Cannabis plant material has been identified to have factors that are inconsistent with Cannabis plant matter that was previously extracted. The content of table 2 may have been populated based on sensed data and evaluations performed using that sensed data. As such sensor data used to populate table 2 may have included data from one or more of a color/imaging sensor, a weight sensor, a density sensor, a mold sensor, a temperature sensor a humidity sensor, a heat sensor, or an oxygen sensor. Assignments of high, medium, or low metrics may have been determined by one or more sets of program code modules. As such, a quality metric (of high, medium, or low) may have been identified by a quality software module, a damage metric (of high, medium, or low) may have been identified by a damage software module, a theft metric (of high, medium, or low) may have been identified by a theft software module, and a correlation harvest to extraction metric (of high, medium, or low) may have been identified by a correlation harvest to extraction software module. In instances when any of these metrics are at, above or below, a threshold level an alert status may be changed from a “no alert” status to a “yes alert” status. As such, alerts may be generated when any of the quality metric, the damage metric, the theft metric, or the correlation harvest to extraction metric (a correlation/extraction metric) are identified to be at a low level.

TABLE 2
Cutting, Trimming, & Drying Data/Metrics

Record ID

| | |
|---|---|
| 55 | 56 |

Cutting

| | | |
|---|---|---|
| Avg. Color | Green | • |
| Plant | 20% | • |
| Density | | |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert Theft | No | • |
| Corr./Ext | High | • |
| Metric | | |
| Alert Status | No | • |

Wet Trim

| | | |
|---|---|---|
| Avg. | Green | • |
| Color | | |
| Plant | 30% | • |
| Density | | |
| Mold | • | • |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert | No | • |
| Theft | | |
| Corr.\EXT | High | • |
| Metric | | |
| Corr./EXT | No | • |
| Alert | | |

Drying

| | | |
|---|---|---|
| Color | Brown | • |
| Plant | 15% | • |
| Density | | |
| Avg. | 75 | • |
| Temp. | | |
| Average | 40% | • |
| Humidity | | |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert | No | • |
| Theft | | |
| Corr./EXT | High | • |
| Metric | | |
| Corr./EXT | No | • |
| Alert | | |

TABLE 3
De-Stemming, Curing, and Shipping Data/Metrics

Record ID

| | |
|---|---|
| 55 | 56 |

De-Stemming

| | | |
|---|---|---|
| Color | Brown | • |
| Density | 20% | • |
| Mold | No | • |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert | No | • |
| Theft | | |
| Corr./EXT | High | • |
| Metric | | |
| Alert | No | • |
| Status | | |

Curing

| | | |
|---|---|---|
| Color | Brown | • |
| Density | 20% | • |
| Mold | No | • |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert | No | • |
| Theft | | |
| Corr./EXT | High | • |
| Metric | | |
| Alert | No | • |
| Status | | |

Shipping

| | | |
|---|---|---|
| Color | Brown | • |
| Density | 20% | • |
| Mold | No | • |
| Quality | High | • |
| Metric | | |
| Alert | No | • |
| Quality | | |
| Damage | High | • |
| Metric | | |
| Alert | No | • |
| Damage | | |
| Theft | High | • |
| Metric | | |
| Alert | No | • |
| Theft | | |
| Corr./EXT | High | • |
| Metric | | |
| Alert | No | • |
| Status | | |

Table 3 includes data that may have been collected or generated in each of steps 240, 250, and 260 of FIG. 2 when lots of Cannabis plants 55 and 56 were processed. Table 3 includes a first set of data collected or generated during the de-stemming step 240, a second set of data collected or generated during the curing step 250, and a third set of data collected during the shipping step 260 of FIG. 2. The data included in table 3 may be stored in harvesting database 120 of FIG. 1. Sensor data collected during each of these steps may have been collected and evaluated continuously when the content of table 2 was added to table 2. Table 2 includes average colors, plant densities, metrics (e.g. quality, damage, theft, & correlation/extraction metrics), alert levels (e.g. quality, damage, theft, correction/extract alert levels), and alert status. Note that levels assigned to the quality metric, the damage metric, the theft metric, and the correlation/extraction (Corr./EXT) metric may have a value of high, medium, or low, for example. A high metric may identify that a plant quality, an amount of damage, an amount of theft, or and expected extraction efficiencies for a given lot of Cannabis plant material are each at a preferred level. As such, high metrics may identify that the Cannabis plant material is at a high quality level, an amount of damage is little to none, an amount of theft is little to none, and that the Cannabis plant material has been identified to have factors that are consistent with Cannabis plant matter that was previously extracted. Medium metrics may identify that the Cannabis plant material quality is at a medium level, an amount of damage is modest, an amount of theft is modest, and that the Cannabis plant material has been identified to have factors that are similar to, yet not judged to be completely consistent with Cannabis plant matter that was previously extracted. Low metrics may identify that the Cannabis plant material quality is at a low level, an amount of damage is above a threshold level, an amount of theft is above a threshold level, and that the Cannabis plant material has been identified to have factors that are inconsistent with Cannabis plant matter that was previously extracted. The content of table 2 may have been populated based on sensed data and evaluations performed using that sensed data. As such sensor data used to populate table 2 may have included data from one or more of a color/imaging sensor, a weight sensor, a density sensor, a mold sensor, a temperature sensor a humidity sensor, a heat sensor, or an oxygen sensor. Assignments of high, medium, or low metrics may have been determined by one or more sets of program code modules. As such, a quality metric (of high, medium, or low) may have been identified by a quality software module, a damage metric (of high, medium, or low) may have been identified by a damage software module, a theft metric (of high, medium, or low) may have been identified by a theft software module, and a correlation harvest to extraction metric (of high, medium, or low) may have been identified by a correlation harvest to extraction software module. In instances when any of these metrics are at, above, or below, a threshold level an alert status may be changed from a “no alert” status to a “yes alert” status. As such, alerts may be generated when any of the quality metric, the damage metric, the theft metric, or the correlation harvest to extraction metric are identified to be at a low level.

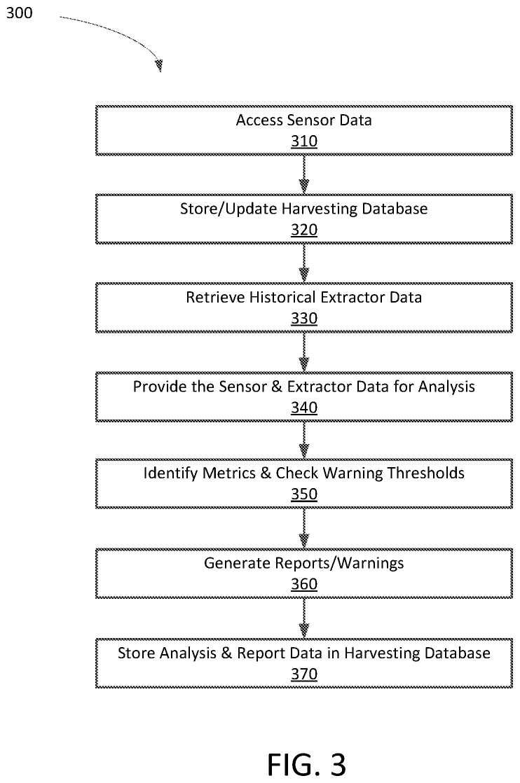

FIG. 3 is a flowchart illustrating an exemplary method 300 for tracking harvest data. The method 300 of FIG. 3 may be performed by harvesting platform server 105 and may begin with step 310 where sensor data may be accessed. The harvesting base software module may pull new data from sensors and may store that data in into a harvesting database in step 320. Next, the base software may cause the harvesting platform server 105 of FIG. 1 to retrieve historical data stored at extractor database in step 330 of FIG. 3. The collection of the new sensor data may trigger the harvesting base module program code to send communications to and receive data from extraction network computer 150 using API 110, communication interface 115, communication interface 155, and API 165. Here again these communications may pass through cloud or Internet 170 of FIG. 1. The base module may then provide the sensors data and the extractor historical database to an analysis software module at step 340 of FIG. 3. The analysis module may then send back to the harvesting base module metrics that were identified or estimated by the analysis software module or data related to any warnings or alerts that could be issued in step 350 of FIG. 3. The harvesting base module may then generate a report or a message to notify a harvester and/or an extractor of any issues detected by operation of the analysis software module in step 360. The generated reports or messages may then be sent to computing devices of a grower or an extractor. The harvesting base module may then store the information received from analysis module and any reports or warning message data in the harvesting database at step 370 of FIG. 3.

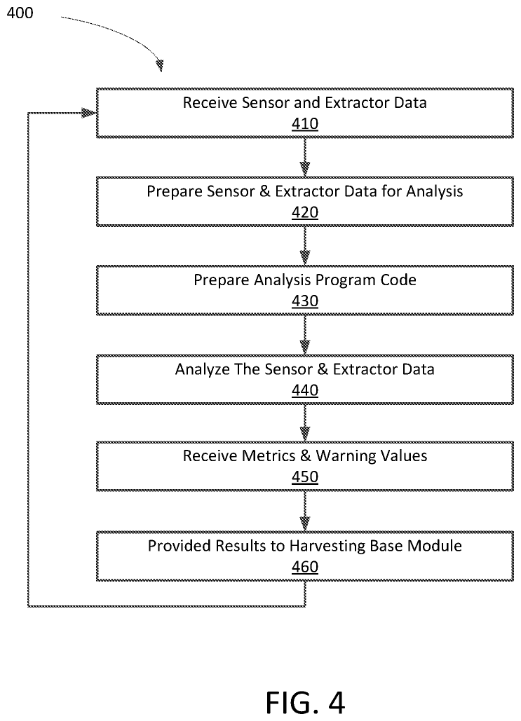

FIG. 4 is a flowchart illustrating an exemplary method 400 for harvest data analytics. The method 400 of FIG. 4 begins with step 410 where sensor data and extractor data may be received from the base software module of FIG. 3. Next, in step 420, the received sensor and extractor data may be prepared for analysis in step 430 to identify plant quality, an amount of plant damage, an amount of theft, or to identify whether received sensor data is consistent with historical extraction data. Next, the sensor and analysis data may be analyzed in step 440 of FIG. 4. A processor executing program code of the analysis software module may then execute functions consistent with a quality module, a damage module, a theft module; and a correlation harvest to extractor module. Then, the analysis module may receive a quality metric and warning status (Y/N) from the quality module, a damage metric and warning status (Y/N) from the damage module, a theft metric and warning status (Y/N) from the theft module, and a correlation harvest to extractor metric and warning status (Y/N) from the correlation harvest to extractor module at step 450 of FIG. 4. After step 450, the analysis module may send the metrics and warning status to the harvesting base software module in step 460 of FIG. 4. After step 460, program flow may flow back to step 410 of FIG. 4, where additional sensor data may be received.

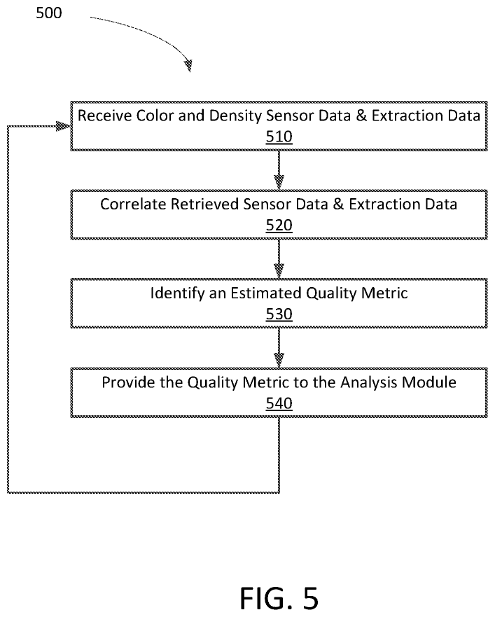

FIG. 5 is a flowchart illustrating an exemplary method 500 for identifying a quality of a set of Cannabis plant matter. Such method 500 may result from execution of the quality software module. Step 510 of FIG. 5 may receive sensor data and extraction data from the base software module. The sensor data received in step 510 may include color data and density data. The Extraction data received in FIG. 5 may include data that identifies a type of Cannabis plant or may include an efficiency rate. The analysis software module may identify whether the sensor data corresponds to the extraction data in step 520 of FIG. 5. The quality software module may compare the current color and density with the values of successful runs in the extractor database (i.e. high efficiency rates) for the type of biomass being used. The quality software module may then estimate a quality metric (e.g. of high, medium, or low) based on the previous correlation analysis and extraction results in step 530. Next, in step 540 the quality metric may be provided to the analysis software module. A warning status of yes or no (Y/N) may also be assigned to the plant matter in step 540 of FIG. 5. The quality metric or the warning status provided to the analysis module may result in a warning message to be sent to computers of a grower or an extractor and may result in the quality metric and warning status information to be stored in a harvesting database. After step 540, program flow may move to step 510, where the steps of FIG. 5 may be repeated continuously.

As an example, the temperature and humidity of each stage of the harvesting platform may affect the quality of the Cannabis biomass and therefore temperature and humidity data across the six steps in the harvesting/post-harvesting process is used by the quality module. The temperature keeps always around 68 F (20 C) and the humidity is around 45%. The extractor historical database indicates that when Temperature keeps around 68 F and humidity is around 45% across each of the steps, the efficiency of the extraction is high and therefore, the quality metric may be high, and no warning may need to be generated. The extractor historical database indicates that if the temperature is higher than 80 F or lower than 40 F or the humidity is above 70% or lower than 20% a warning sign should be generated for the harvester to flag that batch and the quality metric should be low. The quality module sends the quality metric and warning status to the analysis module.

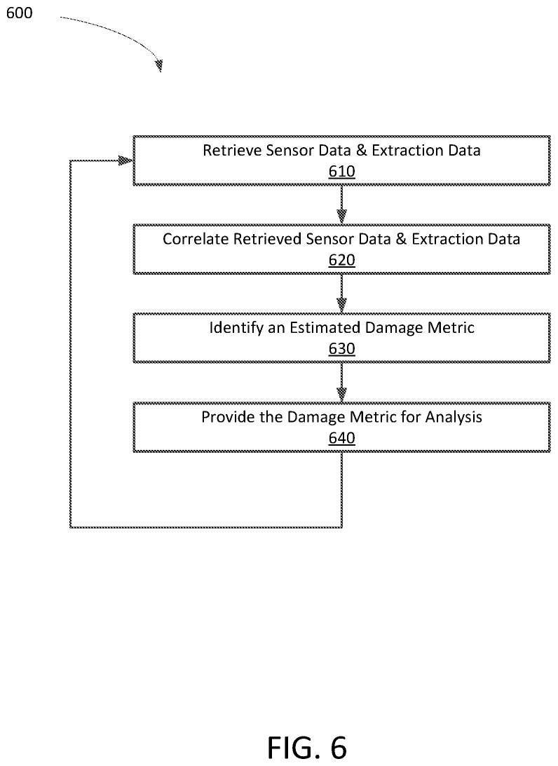

FIG. 6 is a flowchart illustrating an exemplary method 600 for identifying a damage metric of a set of Cannabis plant matter. Method 600 may result from execution of the damage software module. Step 610 of FIG. 6 may receive sensor data and extraction data from the base software module of FIG. 3. The sensor data received in step 610 may include mold sensor data or other data. The Extraction data received in FIG. 6 may include data that identifies a type of Cannabis plant or may include an efficiency rate. The damage software module may identify whether the sensor data corresponds to the extraction data in step 620 of FIG. 6. The quality software module may compare current sensor data (e.g. mold sensor data) with the values of successful runs in the extractor database (i.e. high efficiency rates) for the type of biomass being used. The quality software module may then estimate a damage metric (e.g. of high, medium, or low) based on the previous correlation analysis and extraction results in step 630. Next, in step 640 the damage metric may be provided to the analysis software module. A warning status of yes or no (Y/N) may also be assigned to the plant matter in step 640 of FIG. 6. The damage metric or the warning status provided to the analysis module may result in a warning message to be sent to computers of a grower or an extractor and may result in the damage metric and warning status information to be stored in a harvesting database. After step 640, program flow may move to step 610, where the steps of FIG. 6 may be repeated continuously.

As an example, mold is one parameter that could damage the flowers and tends to happen often during the wet trimming process. Therefore, the damage module receives mold sensor data from the wet trimming phase and the damage module calculates an average mold grow rate with a value of 3 mm/day. The extractor historical database indicates that when mold grow rate in the wet trimming phase is higher than 5 mm/day, the efficiency of the extraction goes down; if the rate is between 2 mm/day and 5 mm/day, the efficiency is average; and if the grow rate is below 2 mm/day, the efficiency is higher. That means that the damage metric for this example may be medium and so the wet trimming can proceed, and no warning needs to be sent. The damage module may then send the damage metric and warning status to the analysis module.

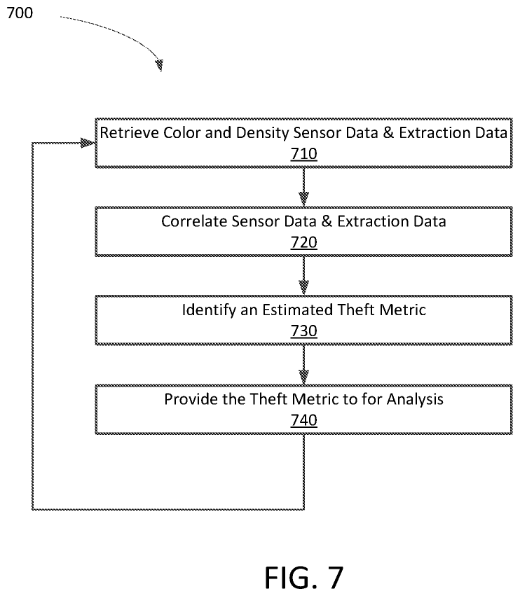

FIG. 7 is a flowchart illustrating an exemplary method 700 for identifying a theft metric of a set of Cannabis plant matter. Method 700 may result from execution of the theft software module. Step 710 of FIG. 7 may receive sensor data and extraction data from the base software module of FIG. 3. The sensor data received in step 710 may include weight data. The Extraction data received in FIG. 7 may include data that identifies a type of Cannabis plant or may include an efficiency rate. The analysis software module may identify whether the sensor data corresponds to the extraction data in step 720 of FIG. 7. The quality software module may compare current sensor data (e.g. the weight data) to the values of successful runs in the extractor database (i.e. high efficiency rates) for the type of biomass being used. The quality software module may then estimate a theft metric (e.g. of high, medium, or low) based on the previous correlation analysis and extraction results in step 730. Next, in step 740 provide the theft metric to the analysis software module. A warning status of yes or no (Y/N) may also be assigned to the plant matter in step 740 of FIG. 7. The theft metric or the warning status provided to the analysis module may result in a warning message to be sent to computers of a grower or an extractor and may result in the theft metric and warning status information to be stored in a harvesting database. After step 740, program flow may move to step 710, where the steps of FIG. 7 may be repeated continuously.

As an example, theft may happen between the curing and the shipping steps. The theft module receives weight sensor data at the end of the curing process and at the beginning of the shipment. The theft module calculates the average % of weight lost in the transfer—0.3% in this example. The extractor historical database indicates that 0.5% of weight loss may be expected during transfer from curing to shipping and that that loss would not affect the efficiency of the extraction. Therefore, the theft metric should be high, and no warning should be generated. The theft module sends the theft metric and warning value to the analysis module.

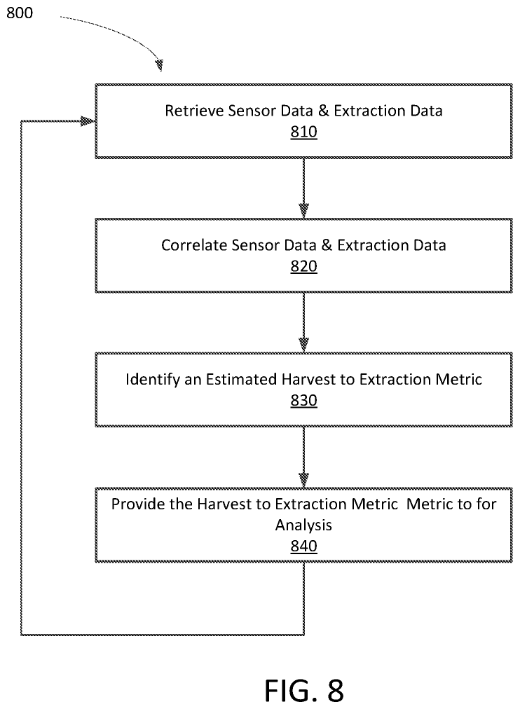

FIG. 8 is a flowchart illustrating an exemplary method 800 for identifying a extraction metrics of a set of Cannabis plant matter. Method 800 may result from execution of the correlation harvest to extraction software module. Step 810 of FIG. 8 may receive sensor data and extraction data from the base software module of FIG. 3. The sensor data received in step 810 may include color data. The Extraction data received in FIG. 8 may include data that identifies a type of Cannabis plant or may include an efficiency rate. The analysis software module may identify whether the sensor data corresponds to the extraction data in step 820 of FIG. 8. The quality software module may compare current sensor data (e.g. the color data) to the values of successful runs in the extractor database (i.e. high efficiency rates) for the type of biomass being used. The quality software module may then estimate a correlation/extraction metric (e.g. of high, medium, or low) based on the previous correlation analysis and extraction results in step 830. Next, in step 840 provide the theft metric to the analysis software module. A warning status of yes or no (Y/N) may also be assigned to the plant matter in step 840 of FIG. 8. The correlation/extraction metric or the warning status provided to the analysis module may result in a warning message to be sent to computers of a grower or an extractor and may result in the correlation/extraction metric and warning status information to be stored in a harvesting database. After step 840, program flow may move to step 810, where the steps of FIG. 8 may be repeated continuously.

As an example, changes in color during curing may indicate over curing. The correlation harvest to extraction module receives color data from a hyperspectral camera placed at the curing step and correlates that data with data found in the extractor historical database found for similar biomass during the curing process. The historical database indicates that for similar biomass, colors in the green spectrum during curing show a decrease in extraction efficiency and therefore, if the data from the hyperspectral camera shows the color of the Cannabis biomass falling into the green range of wavelengths, a “Yes” status or value may be assigned to the warning parameter and the harvesting base module would generate a warning for the harvester to stop the curing process. The correlation harvest to extraction module sends the correlation harvest to extraction metric and warning value to the analysis module.

Table 4 includes data that may be used to identify preferred parameters for extracting cannabinoids from specific types of Cannabis plant matter. Parameters stored in table 4 may be settings that affect how an extraction system operates. Such parameters may identify a solvent type, microwave energy level, extraction temperatures, a ratio of solvent volume to plant mass, or extraction time (e.g. length of time that Cannabis biomass resides (residence time) in a continuous flow extraction chamber. Note that table 3 includes each of these parameters. The data of table 4 may also be used to cross-reference different Cannabis types with different biomass lot numbers, with different extraction parameters, and with different extract potencies and efficiency rates. Table 4 includes three different lots of high THC Cannabis, two different lots of high CBD Cannabis and two different lots of low THC hemp. A higher efficiency rate in table 3 for a given Cannabis plant biomass type may be used to identify a preferred set of parameters for extracting cannabinoids included in a given type of Cannabis plant biomass. The high THC Cannabis of lot number T104 was assigned a 98% efficiency rate used parameters of Low microwave energy density and 12 minutes of residence time when ethanol was used as an extraction solvent at a ratio of 12 liters per kg. High THC Cannabis lots T001 and T002 resulted in lower efficiency rates. As such, high THC Cannabis lot T104 had the best extraction efficiency rate. Similarly, high CBD Cannabis lot C220 had the highest extraction efficiency rate and low THC hemp lot H001 had the highest efficiency rate. Analysis program code consistent with the present disclosure may compare extraction efficiency rates as reviewed above and preferred extraction process parameters may be selected for a new lot of Cannabis plant biomass in step 350 based on the comparison of the efficiency factors. As such, when the new lot of Cannabis plant biomass is high THC Cannabis, extraction parameters used to extract Cannabis lot T104 may be used to extract the new lot of Cannabis plant biomass because historically, those parameters resulted in best extraction efficiencies for high THC Cannabis. Alternatively, when the new lot of Cannabis plant material is low THC hemp, extraction parameters consistent with Cannabis plant material lot H001 may be selected because historically, those parameters resulted in best extraction efficiencies for low THC hemp.

Each of these different efficiencies may be compared and preferred extraction process parameters may be identified by reviewing historical data. In certain instances, data stored at extraction network computer 150 of FIG. 1 may include data from different extractors that use different extraction methods that may also require different process parameters. In certain instances, different extractors may use processes that are similar, yet use different solvents. Finally, yield projections, preferred extraction process parameters, and efficiency rates data may be identified sent to computers of a grower that is growing the Cannabis. This data could help the grower to negotiate a contract to sell concentrates made from their plant matter.

TABLE 4
Cannabis Extraction Process Parameters vs. Process Efficiencies

| | | | | Solvent to | | |
|---|---|---|---|---|---|---|
| Cannabis | | | Microwave | Biomass | Residence | |
| Biomass | Lot Number/Biomass | | Power | Ratio | Time | Efficiency |
| Type | Reference Number | Solvent | Density | (l/kg) | (min) | Rate |
| High THC | T001 | Ethanol | Low | 10 | 5 | 93% |
| Cannabis | | | | | | |
| High THC | T002 | Pentane | Med | 10 | 5 | 97% |
| Cannabis | | | | | | |
| High THC | T104 | Ethanol | Low | 12 | 20 | 98% |
| Cannabis | | | | | | |
| High CBD | C220 | Ethanol | High | 12 | 10 | 94% |
| Cannabis | | | | | | |
| High CBD | C301 | Ethanol | Low | 12 | 10 | 87% |
| Cannabis | | | | | | |
| Low THC | H001 | Ethanol | Med | 12 | 20 | 83% |
| Hemp | | | | | | |
| Low THC | H202 | Ethanol | Low | 10 | 20 | 71% |
| Hemp | | | | | | |

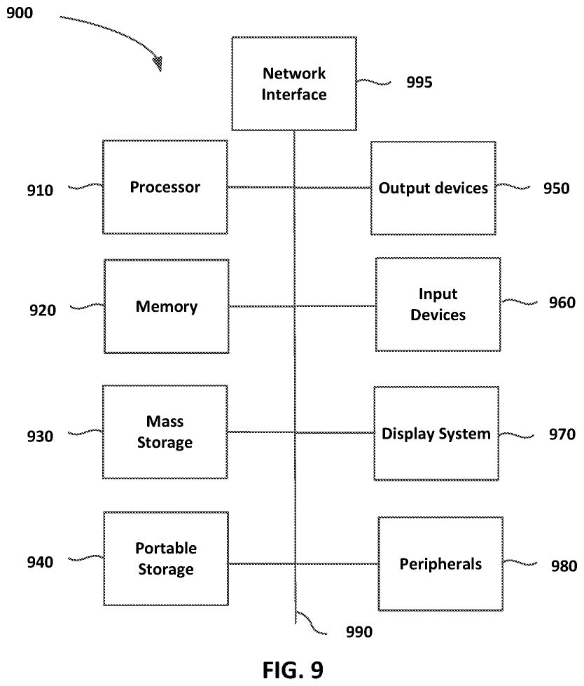

FIG. 9 illustrates a computing system that may be used to implement an embodiment of the present invention. The computing system 900 of FIG. 9 includes one or more processors 910 and main memory 920. Main memory 920 stores, in part, instructions and data for execution by processor 910. Main memory 920 can store the executable code when in operation. The system 900 of FIG. 9 further includes a mass storage device 930, portable storage medium drive(s) 940, output devices 950, user input devices 960, a graphics display 970, peripheral devices 980, and network interface 995.

The components shown in FIG. 9 are depicted as being connected via a single bus 990. However, the components may be connected through one or more data transport means. For example, processor unit 910 and main memory 920 may be connected via a local microprocessor bus, and the mass storage device 930, peripheral device(s) 980, portable storage device 940, and display system 970 may be connected via one or more input/output (I/O) buses.

Mass storage device 930, which may be implemented with a magnetic disk drive or an optical disk drive, is a non-volatile storage device for storing data and instructions for use by processor unit 910. Mass storage device 930 can store the system software for implementing embodiments of the present invention for purposes of loading that software into main memory 920.

Portable storage device 940 operates in conjunction with a portable non-volatile storage medium, such as a FLASH memory, compact disk or Digital video disc, to input and output data and code to and from the computer system 900 of FIG. 9. The system software for implementing embodiments of the present invention may be stored on such a portable medium and input to the computer system 900 via the portable storage device 940.

Input devices 960 provide a portion of a user interface. Input devices 960 may include an alpha-numeric keypad, such as a keyboard, for inputting alpha-numeric and other information, or a pointing device, such as a mouse, a trackball, stylus, or cursor direction keys. Additionally, the system 900 as shown in FIG. 9 includes output devices 950. Examples of suitable output devices include speakers, printers, network interfaces, and monitors.

Display system 970 may include a liquid crystal display (LCD), a plasma display, an organic light-emitting diode (OLED) display, an electronic ink display, a projector-based display, a holographic display, or another suitable display device. Display system 970 receives textual and graphical information, and processes the information for output to the display device. The display system 970 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripherals 980 may include any type of computer support device to add additional functionality to the computer system. For example, peripheral device(s) 980 may include a modem or a router.

Network interface 995 may include any form of computer interface of a computer, whether that be a wired network or a wireless interface. As such, network interface 995 may be an Ethernet network interface, a BlueTooth™ wireless interface, an 802.11 interface, or a cellular phone interface.

The components contained in the computer system 900 of FIG. 9 are those typically found in computer systems that may be suitable for use with embodiments of the present invention and are intended to represent a broad category of such computer components that are well known in the art. Thus, the computer system 900 of FIG. 9 can be a personal computer, a hand held computing device, a telephone (“smart” or otherwise), a mobile computing device, a workstation, a server (on a server rack or otherwise), a minicomputer, a mainframe computer, a tablet computing device, a wearable device (such as a watch, a ring, a pair of glasses, or another type of jewelry/clothing/accessory), a video game console (portable or otherwise), an e-book reader, a media player device (portable or otherwise), a vehicle-based computer, some combination thereof, or any other computing device. The computer can also include different bus configurations, networked platforms, multi-processor platforms, etc. The computer system 900 may in some cases be a virtual computer system executed by another computer system. Various operating systems can be used including Unix, Linux, Windows, Macintosh OS, Palm OS, Android, iOS, and other suitable operating systems.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASH EPROM, and any other memory chip or cartridge.

One skilled in the art may appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure describes various embodiments of systems, methods, and various other aspects of the disclosure. Any person with ordinary skills in the art may appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

What is claimed is:

1. A method for monitoring the processing of plant biomass, the method comprising:
storing extraction data in an extraction database in memory regarding at least one past Cannabis biomass, the extraction data indicative of a threshold;
receiving sensor data from one or more sensors associated with different processing stages of a current Cannabis biomass;
generating one or more metrics based on the received sensor data;
comparing the generated metrics to the extraction data, wherein at least one of the metrics is indicative of a level of cannabinoid extraction efficiency; and
sending a warning message to a computing device when the metric indicative of the cannabinoid extraction efficiency level at least meets the threshold.

2. The method of claim 1, further comprising initially identifying that the metrics are consistent with the extraction data.

3. The method of claim 1, wherein the generated metrics are indicative of a quality level of the current Cannabis biomass.

4. The method of claim 1, wherein the threshold is indicative of a damage level or a theft likelihood of at least a portion of the current Cannabis biomass.

5. The method of claim 1, further comprising storing the received sensor data in a harvesting database in memory.

6. The method of claim 1, further comprising storing the one or more metrics at a harvesting database in memory.

7. The method of claim 1, wherein the extraction data further includes a set of extraction parameters used for extracting the at least one past Cannabis biomass.

8. The method of claim 7, further comprising identifying one or more extraction parameters for extracting cannabinoids from the current Cannabis biomass based on the extraction parameters used to extract the at least one past Cannabis biomass.

9. The method of claim 1, wherein comparing the generated metrics to the extraction data includes retrieving the extraction data from the extraction database, and wherein retrieving the extraction data is based on a correlation to the at least one past Cannabis biomass.

10. A system for monitoring the processing of plant biomass, the system comprising:
an extraction database in memory that stores extraction data regarding at least one past Cannabis biomass, the extraction data indicative of a threshold;
one or more sensors that capture data regarding different processing stages of a current Cannabis biomass; and
a harvesting server that:
generates one or more metrics based on the received sensor data,
compares the generated metrics to the extraction data, wherein at least one of the metrics is indicative of a level of cannabinoid extraction efficiency, and
sends a warning message to a computing device when the metric indicative of the cannabinoid extraction efficiency level at least meets the threshold.

11. The system of claim 10, wherein the harvesting server initially identifies that the metrics are consistent with the extraction data.

12. The system of claim 10, wherein the generated metrics are indicative of a quality level of the current Cannabis biomass.

13. The system of claim 10, wherein the threshold is indicative of a damage level or a theft likelihood of at least a portion of the current Cannabis biomass.

14. The system of claim 10, further comprising a harvesting database in memory that stores the received sensor data.

15. The system of claim 10, further comprising storing the one or more metrics at a harvesting database in memory.

16. The system of claim 10, wherein the extraction data further includes a set of extraction parameters used for extracting the at least past Cannabis biomass.

17. The system of claim 16, wherein the harvesting platform further identifies one or more extraction parameters for extracting cannabinoids from the current Cannabis biomass based on the extraction parameters used to extract the at least one past Cannabis biomass and sends the identified extraction parameters to an extraction apparatus.

18. The system of claim 10, wherein the harvesting server compares the generated metrics to the extraction data by retrieving the extraction data from the extraction database based on a correlation to the at least one past Cannabis biomass.

19. A non-transitory, computer-readable storage medium, having embodied thereon a program executable by a processor to perform a method for monitoring the processing of plant biomass, the method comprising:
storing extraction data in an extraction database in memory regarding at least one past Cannabis biomass, the extraction data indicative of a threshold;
receiving sensor data from one or more sensors associated with different processing stages of a current Cannabis biomass;
generating one or more metrics based on the received sensor data;
comparing the generated metrics to the extraction data, wherein at least one of the metrics is indicative of a level of cannabinoid extraction efficiency; and
sending a warning message to a computing device when the metric indicative of the cannabinoid extraction efficiency level at least meets the threshold.
